(19) United States
(12) Patent Application Publication
(10) Pub. No.: US 2026/0240674 A1
(43) Pub. Date: Aug. 20, 2026

(54) HAND ORTHOSIS OR HAND PROSTHESIS, AND SYSTEM CONSISTING OF A HAND ORTHOSIS OR HAND PROTHESIS AND COUNTER ELEMENT

(71) Applicant: OTTOBOCK SE & CO. KGAA, Duderstadt (DE)

(72) Inventors: Viktor Gerhard HÖRIG, Duderstadt (DE); Johas BORNMANN, Duderstadt (DE); Pascal RHEINLÄNDER, Duderstadt (DE)

(21) Appl. No.: 19/471,590

(22) PCT Filed: Apr. 3, 2024
(86) PCT No.: PCT/EP2024/059070

§ 371 Date: Oct. 1, 2025

(30) Foreign Application Priority Data
Apr. 4, 2023 (DE) 10 2023 108 580.8

(51) Int. Cl. A61F 5/01

(52) U.S. Cl. CPC A61F 5/013; A61F 2005/0197

(57) ABSTRACT

The invention relates to a hand orthosis or hand prosthesis having a support part (20) and having at least one finger portion (30), which is arranged distally on the support part (20) and is mounted pivotably relative to the support part (20), the finger portion (30) being formed segmentally from a plurality of finger parts (34, 36, 38) that are mounted articulated to one another, wherein a coupling rod (80) is coupled to a distal finger part (36, 38) and to a proximal finger part (34, 36) or to the support part (20) while crossing two joints and so as to transmit force, the coupling rod (80) being mounted in one case on the palmar side and in the other case dorsally with respect to the respective pivot axis (35, 37) of the joints.

The invention relates to a hand orthosis or hand prosthesis having a support part and having at least one finger portion, which is arranged distally on the support part and is mounted pivotably relative to the support part, the finger portion being formed segmentally from a plurality of finger parts that are mounted articulated to one another, and to a system consisting of such a hand orthosis or hand prosthesis and a counter element.

Hand orthoses are orthoses which are arranged at least partially on the hand and by which it is possible to influence movements of the entire hand relative to the forearm and/or finger movements. This may for example be done by supporting particular movements or opposing particular movements with a resistance. Particular movements may likewise be blocked or the phalanges or extremities may be guided relative to one another via an orthosis.

Hand prostheses replace hands which are not present, or no longer present, and besides a purely cosmetic effect should also partially replace functions of a natural hand. Hand prostheses or prosthetic hands have a base body or a support part, on which at least one artificial finger or finger portion is distally arranged. Often, a thumb element is arranged on the base body and provides a component lying opposite the finger element or the artificial fingers. A finger portion or prosthetic finger may be arranged mobile, in particular articulated on the support part and be displaced via an actuator. There are motor-driven prosthetic hands in which the drive is arranged as an electric motor inside the support part or the base body. There are furthermore prosthetic hands with a grip function driven via a control wire, in which the control wire is secured for example to the patient's shoulder.

DE 20 2015 008 342 U1 relates to a hand orthosis having a forearm buckle and a metacarpal buckle connected to the forearm buckle via a connecting piece and has at least one flexurally elastic element that is fastened to the metacarpal buckle and runs essentially in the distal-proximal direction, and one fastening element respectively arranged in the region of the distal end of the elastic element for fastening a finger. Such a hand orthosis has the effect that, because of the flexurally elastic element which extends along the finger, a restoring force active on the finger is built up when the finger is deflected from a predetermined neutral position by flexion or extension. Such an orthosis may in particular be advantageous for partial paralyses which can result for example from injuries or a stroke.

U.S. Pat. No. 9,861,500 B2 discloses a prosthetic grip unit having a base body and two finger parts, which are mounted pivotably with respect to one another on the base body. An electric motor is arranged inside the base body in order to drive one of the two finger parts. An actuation lever is likewise arranged on the base body and is actuated via a Bowden cable.

DE 10 2017 130 082 B4 discloses a prosthetic hand having a thumb element and at least one finger element. The thumb element is displaced by a control force relative to the one finger element, and is movable along a first trajectory in a first mode and along a second trajectory in a second mode. The prosthetic hand has a force application element that applies a restoring force, which is directed opposite to the control force, to the thumb element. The control force is applied via a tension element, which is arranged on a shoulder or a torso of the wearer of the prosthetic hand.

EP 2 512 758 B1 relates to an apparatus having a first segment and a second segment, the first segment being coupled between a first and a second joint. The first joint is designed for coupling to a base element in order to enable joint mobility of the first element in relation to the base element. A first differential is configured to receive torque and to transmit torque to the first and the second joints. A stop limits the rotation of the second joint. A stop connecting piece is arranged between the first joint and the stop in order to move the stop based on the degree of rotation of the first joint. In order to be able to displace the first segment, a drive is provided that pivots a pivot part, which is arranged pivotably on the first segment, via a first connecting rod. Arranged on the opposite side of the pivot part, there is a second connecting rod which is fastened to the second segment and pivots the second segment relative to the first segment in the event of a displacement of the pivot part.

It is an object of the present invention to provide an improved hand orthosis or hand prosthesis, which facilitates daily tasks.

This object is achieved by a hand orthosis or hand prosthesis having the features of the main claim. Advantageous configurations and developments of the invention are disclosed in the dependent claims, the description and the figures.

The hand orthosis or hand prosthesis having a support part and having at least one finger portion, which is arranged distally on the support part, the finger portion being mounted pivotably relative to the support part and being formed segmentally from a plurality of finger parts that are mounted articulated to one another, is distinguished in that a coupling rod is coupled to a distal finger part and to a proximal finger part or to the support part while crossing two joints and so as to transmit force, the coupling rod being mounted in one case on the palmar side and in the other case dorsally with respect to the respective pivot axis of the joints. In this way, in the case of a flexion movement, for example about the metacarpophalangeal joint, a flexion movement of the proximal interphalangeal joint and optionally also of the distal interphalangeal joint will take place simultaneously. A corresponding displacement or movement is optionally performed on the thumb part. This has the advantage that not all finger parts or components of the thumb part need to be subjected separately to a restoring force or modifying force. Furthermore, the movements of the individual finger parts and optionally of the components of the thumb part are coordinated in a straightforward way. For example, the coupling rod is coupled to a distal finger part or to the one distal component and to a proximal finger part, or proximal component, or to the support part while crossing two joints, no modifying force for modifying the proximal joint component being applied directly on the latter via the coupling rod. A modification takes place via a different force introduction point, in which case the proximal joint component may be modified at the same time. By the arrangement of the mounting points of the coupling rod lying on opposite sides of a connecting line between the joint axes, in the case of pivoting of the proximal phalanx relative to the support part, pivoting of the medial phalanx relative to the proximal phalanx can simultaneously take place so that a closing hand with curving fingers can be achieved with only one drive, for example a motor or spring, or a displacement of the proximal phalanx can be achieved, for example by a muscle.

In one configuration, the hand orthosis or hand prosthesis has an actuator which is coupled with its distal end to the finger portion and displaces the finger portion from an initial setting into a use setting, optionally against the elastic restoring force. In one configuration, the actuator is coupled proximally with respect to the support part to a form-fit element, which can be brought reversibly into engagement with a correspondingly configured counter element, the counter element advantageously having a fastening device for securing to the torso, the shoulder or an extremity of the user. The initial setting of the finger portion may be flexed or extended, i.e. it may have bent fingers or straight fingers.

The finger parts in the initial setting are in one embodiment mounted resiliently on one another and/or on the support part, so that after a modification from the initial setting into a use setting, restoring automatically takes place after the modifying force ceases. The configuration of the finger portion with a plurality of finger parts enables use with the hand orthosis that is assimilated to the natural hand movement and finger movement as well as to the natural hand.

In one configuration, the thumb part is also formed segmentally and the individual components of the thumb part are preferentially mounted articulated to one another, in particular fastened articulated to one another in an initial setting against a spring force. From this initial setting, the finger portion or thumb portion is displaced in the case of elastic mounting against the elastic restoring force into the desired use setting. If the finger portion, and in the case of a hand orthosis with fingers fastened to the finger portion also the finger or fingers of that hand, are in the fully extended setting in the initial setting, the use setting is a flexed setting. If the finger portion is in a not fully or maximally flexed setting, the use setting may be occupied both extended and flexed with respect to the initial setting. The actuator enables modification of the finger portion from the initial setting into the use setting, the actuator being any device with which the transition from the initial setting to the use setting is enabled or performed. If a patient has for example paralysis or weakening of the muscles that bring about an extension of the finger, the actuator acts for example in a manner such that an extension is brought about. The finger portion is configured for example to be slightly curved in the initial setting and is moved by the actuator against the elastic restoring force into a stretched or extended setting. In this setting, the hand may be placed on an object and the actuator may be decoupled or deactivated so that the finger portion is moved back into the initial setting by the elastic restoring force. In this initial setting, the object can be held. Alternatively, the initial setting may also be a substantially extended finger setting relative to the back of the hand and the actuator may bring about a flexion that leads to the finger portion, and therefore also the finger secured thereto, being moved into a flexed setting and held in this setting. The flexed setting is then the use setting, for example in order to hold an object. If the actuator is decoupled or deactivated, the finger portion together with the finger fastened thereto returns into the initial setting.

The actuation of the hand orthosis or of the hand prosthesis takes place, for example, by the actuator being coupled via the form-fit element to the counter element. For this purpose the form-fit element, which is fastened to the proximal end of the actuator or forms the proximal end, is brought into engagement with the counter element with a form fit. This is done, for example, by hooking a loop into a hook. The counter element is secured to the torso, the shoulder or an extremity of the user or patient, and forms an opposing bearing for the form-fit element and the actuator. In the simplest form, the actuator is a control wire whose rear, proximal end is provided with a loop or the like which is next put freely on the forearm or the forearm part of a prosthesis and is displaceably configured, and may be fixed to the torso, the shoulder or an extremity. In order to actuate the actuator, the form-fit element or the loop is brought into engagement with the counter element and then the hand is displaced forward, i.e. away from the torso, the shoulder or an extremity and the counter element, so that a tensile force is transmitted via the form-fit element and optionally the force transmission device or the force transmission element onto the actuator, which then displaces the finger portion and/or optionally a thumb part because of the tensile force or a force resulting therefrom or a moment. In order to decouple the actuator, the form-fit element is disengaged from the counter element so that the user again has free mobility of the hand orthosis or hand prosthesis without attachment to the torso, the shoulder or the extremity at the counter element. The free mobility of the hand orthosis or hand prosthesis is restored.

In addition to a finger portion, in one configuration a thumb part is arranged on the support part, which in one configuration is arranged modifiably on the support part. It is therefore possible not only to receive and support fingers or replicate fingers by prosthetic fingers, but also to support the thumb or replicate a thumb. The support part may extend over the forearm so that the thumb part can be fastened to a forearm shell or a wrist mechanism. The thumb part, which in a configuration as a hand orthosis has a thumb receiver, may be mounted on the support part so that it can be fixed in a use setting, for example in order to be able to firmly adjust different types of grip. In this way, for example, it is possible to implement the pulp pinch or pinch grip or also a fist closure with involvement of the thumb. The displacement of the components of the thumb part relative to one another and optionally relative to the support part takes place in particular by the actuator.

The thumb part is in one configuration formed resiliently or elastically, for example when it is formed integrally with the support part or rigidly fastened thereto, or mounted resiliently on the support part. Resilient mounting may, for example, be achieved by articulated mounting with an assigned spring element. The resiliently formed or resiliently mounted thumb part is in one configuration coupled to the actuator, and can be displaced in a similar way to the finger portion from an initial setting into a use setting and fixed therein. The displacement against the elastic restoring force allows restoring into the respective initial setting. Because of the modifiable mounting of the thumb part on the support part, the initial setting can be varied.

In one configuration of the orthosis, one or more of the receivers are arranged or formed on the finger portion. In this way, it is possible to couple one, two, three or all fingers to the finger portion. In a configuration as a prosthesis, one or more finger parts or prosthetic fingers are arranged on the support part. In a similar way to the thumb part formed resiliently or mounted resiliently on the support part, in one configuration the at least one finger portion is formed resiliently or mounted resiliently on the support part and coupled to the actuator. The finger portion is also in one configuration arranged modifiably on the support part and may in a further configuration be fixed in an arbitrary initial setting. Different and freely selectable initial settings may therefore be adjusted not only for the thumb part but also for the finger portion.

According to one development, an adjustment device for adjusting the elastic restoring force is assigned to the finger portion and/or to the thumb part in order to be able to carry out adaptation to the respective patient or the respective intended use. The finger portion and/or the thumb part are in one configuration formed as a leaf spring and/or resiliently mounted on the support part via a spring, for example a leaf spring, a torsion spring, a spiral spring, a coil spring or an elastomer element. The leaf spring may consist of steel, a plastic or a fiber-reinforced composite material, or a combination thereof.

The coupling rod is in one configuration resiliently mounted or resiliently formed, which enables independent displacement of the finger parts or thumb components under external loads. In this way, injuries are avoided and the materials in the hand orthosis or hand prosthesis are protected. The resilient or elastic mounting and/or configuration of the coupling rod may be active in the compression direction and/or tension direction, so that the effective length of the coupling rod may be reversibly increased or reduced. By the special configuration as a pliant coupling rod, which is for example resiliently configured and/or adaptable particularly in a controlled way in its length, an adaptive grip movement is possible. A key behavior is the coupling rod that yields in its length, which compensates for the disadvantage of rigid coupling. In the case of a rigid coupling rod, the rotation movements of the metacarpophalangeal joint (MCP) and proximal interphalangeal joint (PIP) are coupled, which in the case of a blocked MCP joint, when the proximal finger part presses against an object, also suppresses further movement of the PIP and therefore displacement of the distal finger part. By a flexible coupling rod with simultaneous driving and a moment about the PIP axis, the distal finger part may continue to move even when the proximal finger part is blocked in its movement by contact with a (gripped) object. Possible embodiments of an elastic coupling rod are, for example, a curved or curvilinear design (for example sinusoidal) or linear hydraulics or linear pneumatics in a correspondingly small design. By the use of a length limitation, for example a wire or a cable along the coupling rod, or linear hydraulics or pneumatics with a stop, further elongation beyond a defined elongation of the coupling rod starting from an unloaded initial setting may be prevented. A minimum length may conversely also be ensured via a stop, so that the coupling rod behaves as a rigid coupling rod beyond this point. In one configuration, the coupling rod has a length limitation or a linear actuator, so that it is possible to limit the maximum elongation or the maximum shortening of the coupling rod in the case of an elastic or variable-length configuration of the coupling rod. The length limitation may for example be formed by an element that is rigid in tension, flexible or optionally elastic, which is arranged on, in particular fastened to, the coupling rod. The length limitation may for example be a wire or cable, or an articulatedly mounted hinge structure, which prevents a tensile movement from causing an excessive elongation of the coupling rod. As soon as the maximum desired length of the coupling rod is reached, the length limitation acts so that the elastic or resiliently formed or mounted coupling rod acts as a rigid coupling rod after this time, or beyond this state. The same may also be achieved via a linear actuator, which may for example be formed as linear pneumatics or linear hydraulics, optionally with spring elements arranged thereon. The coupling rod is then formed as an assembly linearly displaceable toward one another consisting of at least two components, which in their end setting each bear on a stop and thereby prevent further elongation relative to one another. The coupling rod may be formed segmentally, so that different elastomer elements, spring elements or the like may be arranged on the receivers in order to achieve adaptation to the respectively desired spring behavior of the coupling rod.

The coupling rod is in one configuration configured to be at least locally curved or undulating in order to achieve the resilient or elastic behavior, so that the reversible deforming can be achieved in a simple implementation.

In a further configuration, the coupling rod is assigned at least one deformation element, which is arranged on a finger part and/or on the support part. In the case of a displacement of the finger parts with respect to one another or with respect to the support part, the deformation element engages with the coupling rod and deforms it because of the displacement of the coupling rod relative to the deformation element. In this way, it is possible to produce an adapted spring behavior because of the deformation of the coupling rod as a function of the displacement of the individual components with respect to one another.

The actuator may be rotatably or displaceably arranged or mounted on the hand orthosis, in particular on the support part or alternatively on a forearm receiver. The actuators may be manually operable and may, for example, have a grip element or a form-fit element via which a tensile force, a compressive force or a moment can be applied. The actuator is coupled to the finger portion and optionally to the thumb part, for example connected via a tension element, compression element or another force transmission device or another force transmission element to the finger portion or thumb part, and enables displacement from the initial setting into the use setting.

In one development, the actuator is assigned a damper for damping the restoring movement from the use setting into the initial setting. The damper may be arranged directly on the actuator, for example as a brake, as a hydraulic damper, as a friction clutch or the like. In one variant, the damping rate of the damper is adjustable in order to be able to carry out adaptation to the respective user and the respective intended use.

The actuator is in one configuration coupled to a clutch and to a force accumulator, in particular a spring, in order to facilitate actuation, particularly in order to be able to bring about blocking and release easily. The actuator may be coupled to a winding mechanism or a tensioning device, which can be released in order to liberate a stored force and bring about a displacement of the finger portion or of the thumb part.

In one configuration, the actuator is configured as a replaceable module so that different actuators can be fastened easily to the hand orthosis or hand prosthesis. This modular structure offers the possibility that a conventional hand orthosis or hand prosthesis can be retrofitted with an actuator and a fixing device, so that easy replacement of components and adaptation to the respective purpose of use can take place.

In order to enable positional independence of the actuator from the finger portion, the actuator in one configuration has a force transmission element that transmits tensile forces and/or compressive forces from the actuator onto the assembly to be moved or to be subjected to a force.

The force transmission element is in one configuration assigned a deformation element, which is arranged on the finger part and/or on the support part. The deformation element, which is configured for example as a cam disk, influences the course and the displacement of the force transmission element as a function of the displacement of the finger parts with respect to one another, or with respect to the support part. In this way, it is possible that in the case of a displacement of the proximal finger part relative to the support part, for example, a distal finger part is pivoted more strongly about the pivot axis on the proximal finger part.

In one development, a fixing device by which the finger portion, or the thumb part, is held in the use setting is coupled to the actuator or to the finger portion or to the thumb part. The initial setting may be flexed or extended, i.e. it may have bent fingers or straight fingers. From this initial setting, the finger portion or the thumb part, and in the fitted state also the finger fastened thereto, is displaced against the elastic restoring force into the desired use setting. In the respective use setting, the respectively desired activity may then be carried out or the hand may be placed on an object and the fixing device may be deactivated, so that the finger portion or the thumb part is moved back into the initial setting by the elastic restoring force. The finger portion and optionally also the thumb part is therefore held in the initial setting by the fixing device, without the actuator with the form-fit element still having to be fastened to and in contact with the counter element. As mentioned above, the initial setting may also be an extended setting, the finger portion or the thumb part being flexed by the actuator and held in this position by the fixing device. The flexed setting is then the use setting, for example in order to hold an object. If the fixing device is loosened, the finger portion or the thumb part returns into the initial setting, optionally together with the fingers fastened thereto.

The actuator is in one configuration coupled to the form-fit element in order to bring about a manual displacement of the finger portion, or thumb part. The form-fit element is for example configured as a ring, loop, hook-and-loop fastener part or hook. In particular, a form-fit part is arranged or formed with a stiff actuator portion proximally with respect to the support part and therefore also proximally with respect to the finger portion and thumb part, in order to provide facilitated actuation of the form-fit element and facilitated accessibility.

In a configuration as a hand orthosis, the support part bears on the back of the hand in the fitted state of the hand orthosis, and in particular the support part runs above the back of the hand in the fitted state of the hand orthosis and thus enables the finger portion or the finger portions to be guided dorsally with respect to the fingers, in order to keep the palm of the hand free for the desired activities or tasks. Alternatively, the support part may run around the heel of the hand or at the side of the hand, so that the support part runs laterally next to the hand. With such an embodiment, it is also possible to support a finger portion relative to proximal hand portions or arm portions.

The support part in an embodiment as a hand orthosis has a forearm receiver and in the fitted state protrudes proximally beyond the wrist. Supporting on the forearm allows both reliable securing and effective supporting of the finger portion relative to the support part and force distribution over a large area. The support in the case of a hand prosthesis takes place via the forearm socket or the forearm tube.

The invention also relates to a system consisting of a hand orthosis or hand prosthesis as described above and the counter element, which can be fastened to the torso, the shoulder or an extremity of the user of the hand orthosis or hand prosthesis, and has a fastening device for securing to the torso, the shoulder or an extremity, for example the upper arm. The fastening device may for example be configured as a strap, although the fastening device may likewise have, or be configured as, a buckle or a clasp as well as hooks, buttons, screw fastenings or similar, either in order to carry out direct fastening and securing to the body or in order to be able to carry out securing to fastening means such as belts, buckles, shells, cuffs or the like.

Exemplary embodiments of the invention are explained in more detail below with the aid of the figures, in which:

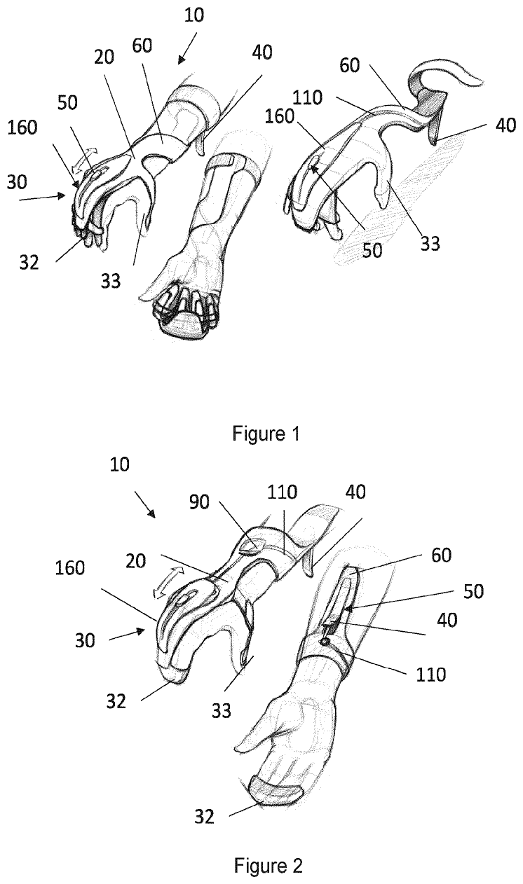

FIG. 1 shows views of two embodiments of a hand orthosis;

FIG. 2 shows two views of a variant of a hand orthosis;

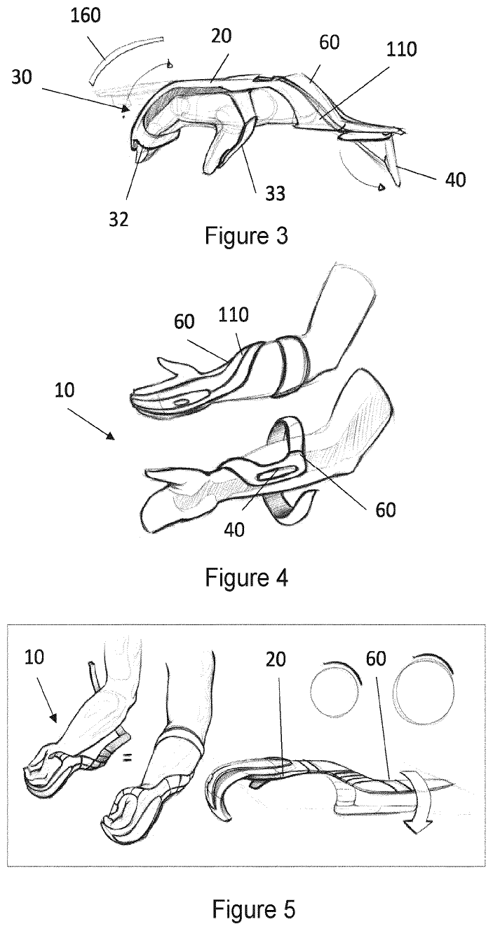

FIG. 3 shows a side view of a hand orthosis according to FIG. 2 in two states;

FIG. 4 shows a variant without a thumb part;

FIG. 5 shows a variant of FIG. 1;

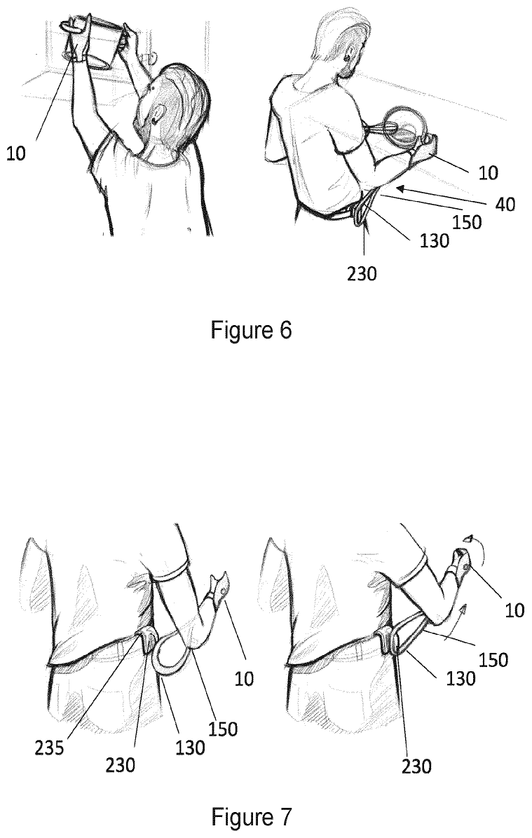

FIG. 6 shows examples of the use of a hand orthosis with an actuator;

FIG. 7 shows representations of the use of a hand orthosis according to FIG. 5;

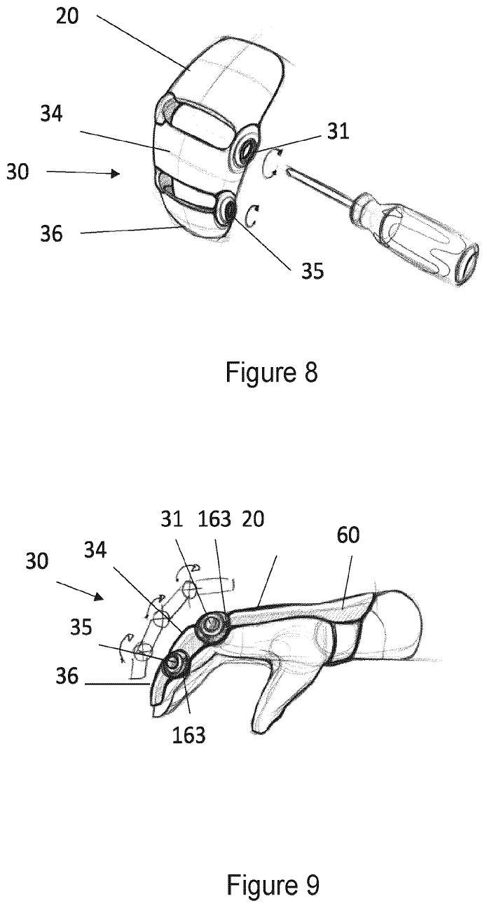

FIG. 8 shows a detail view of a hand orthosis with a segmental finger portion;

FIG. 9 shows a complete representation with a finger portion according to FIG. 7;

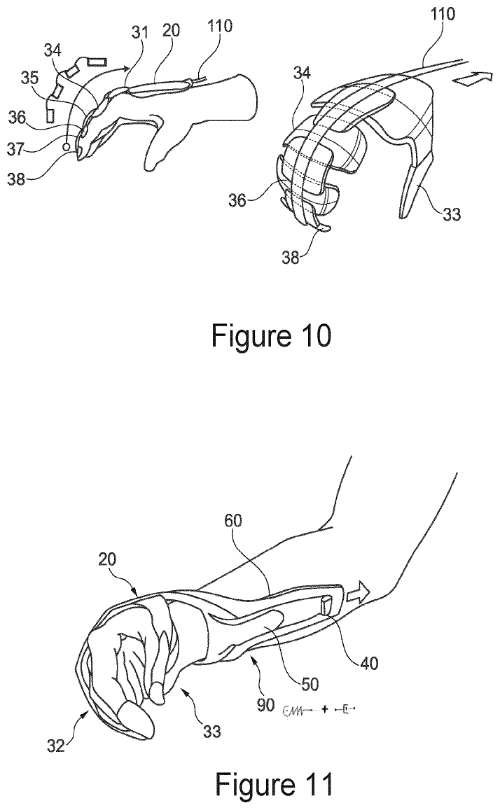

FIG. 10 shows a variant with three finger parts;

FIG. 11 shows a variant with a damped actuator;

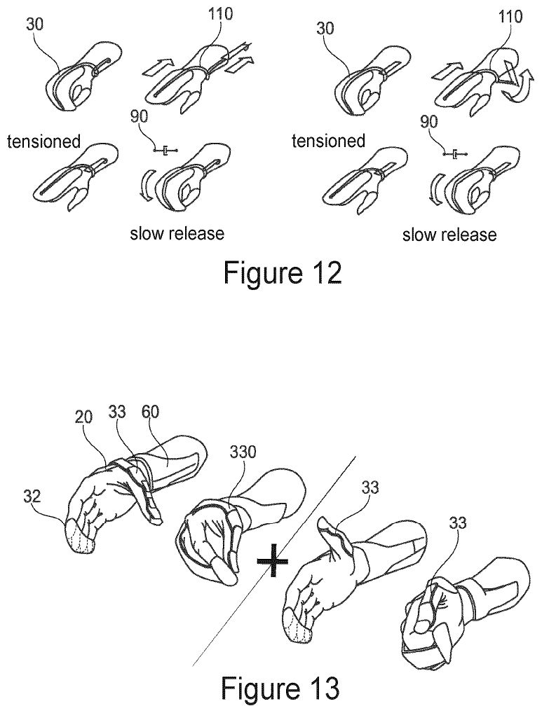

FIG. 12 shows sequential representations of the use of an orthosis;

FIG. 13 shows a variant with a modifiable thumb portion;

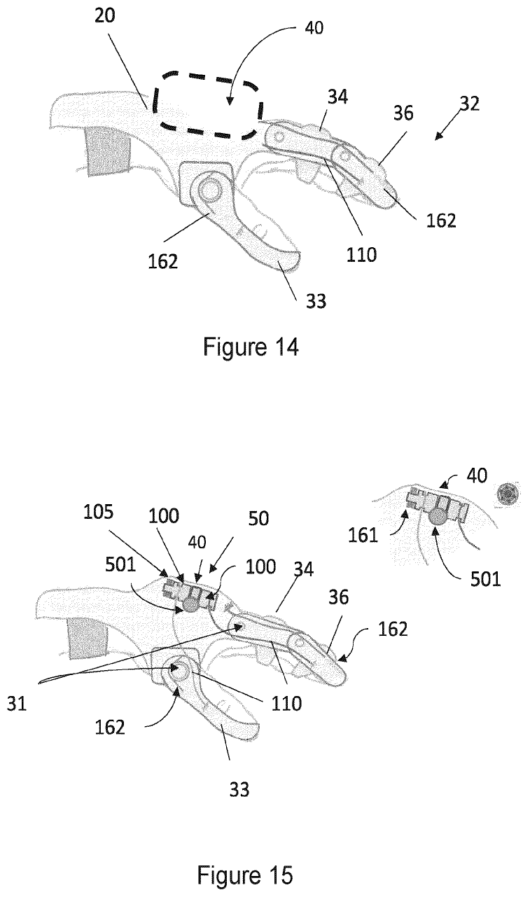

FIG. 14 shows a variant with a configurable actuator;

FIG. 15 shows representations of one embodiment;

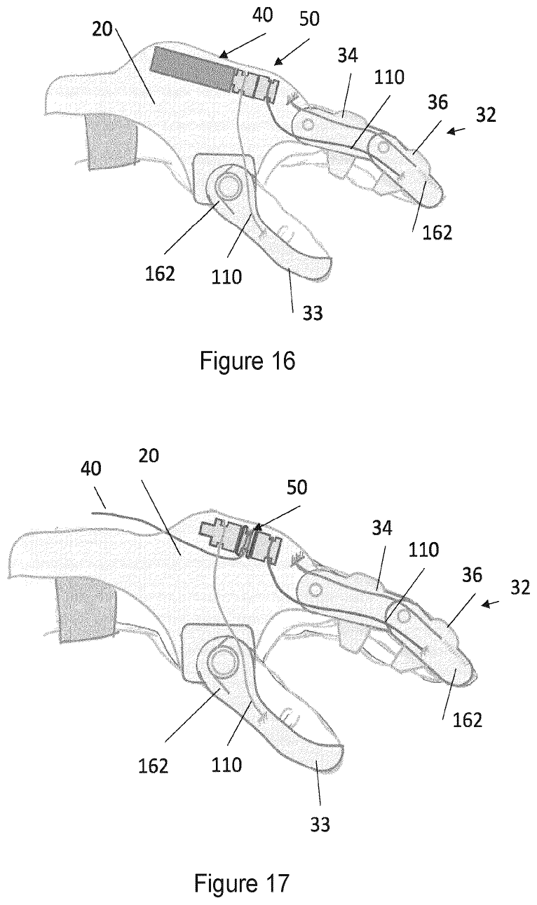

FIG. 16 shows a variant of FIG. 15;

FIG. 17 shows a variant of FIG. 16;

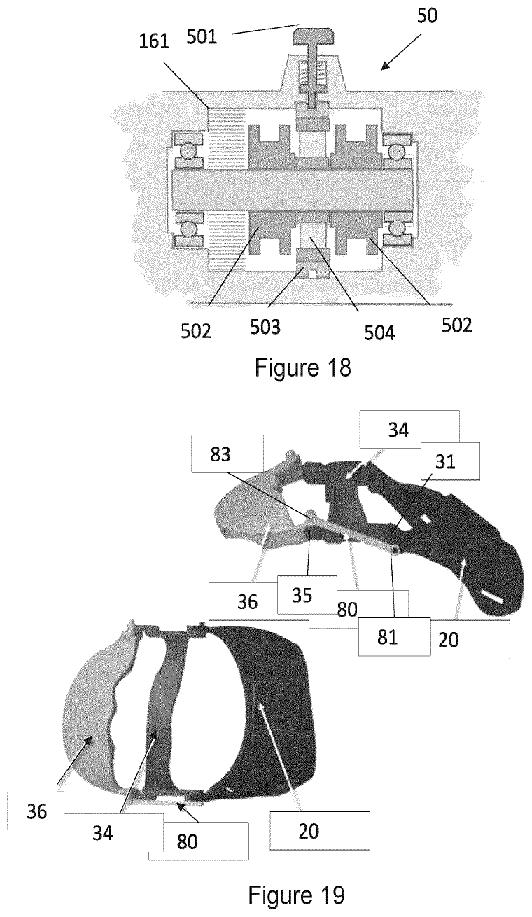

FIG. 18 shows a detail view in a sectional representation of a release mechanism;

FIG. 19 shows a support part with a segmental finger portion and a coupling rod;

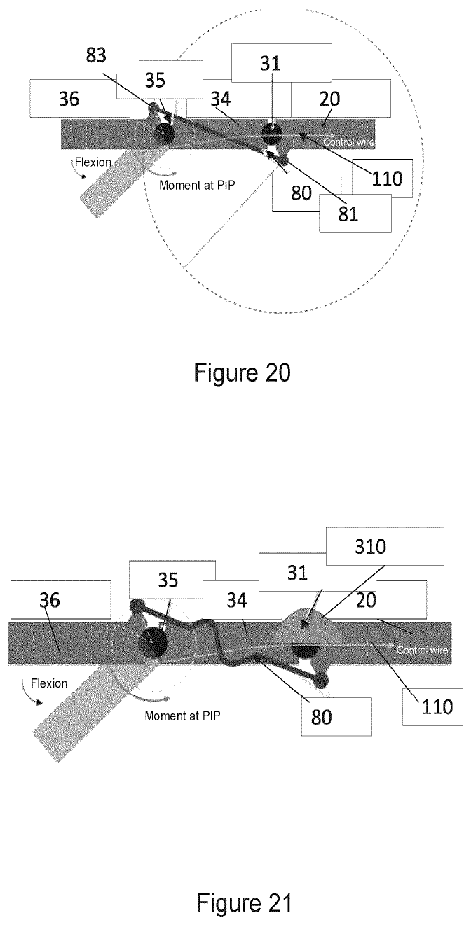

FIG. 20 shows a schematic representation of an embodiment with a deformation element;

FIG. 21 shows a variant of FIG. 20 in a flexed setting;

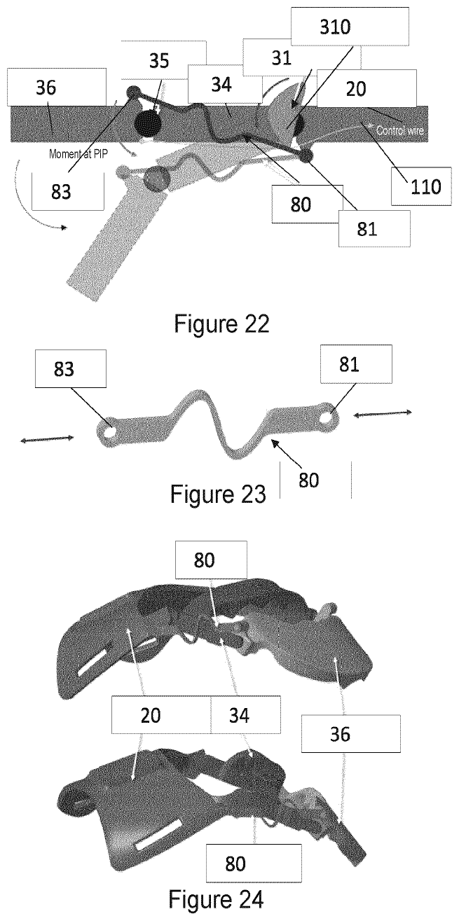

FIG. 22 shows a representation of FIG. 20 during use;

FIG. 23 shows a coupling rod in a detail view;

FIG. 24 shows perspective views of a hand orthosis with a coupling rod according to FIG. 23;

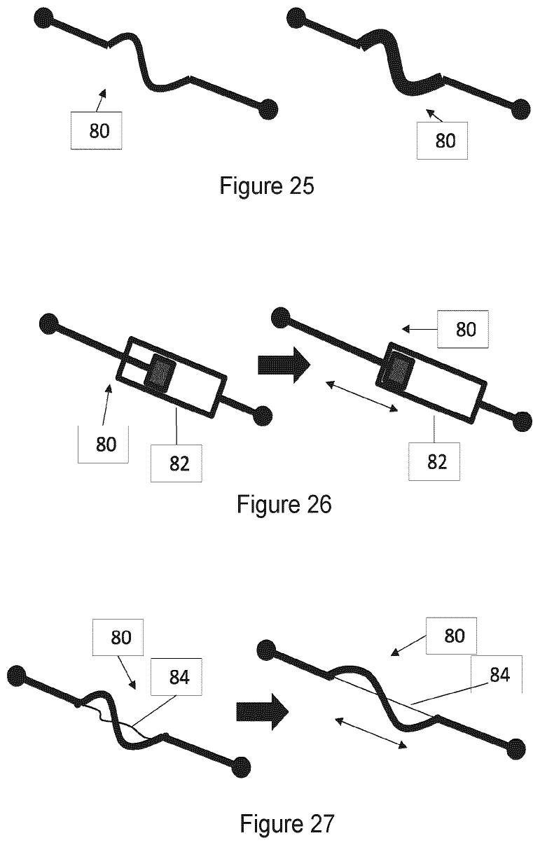

FIG. 25 shows variants of the coupling rod;

FIG. 26 shows representations of a coupling rod with linear actuators;

FIG. 27 shows representations of a coupling rod with a length limitation;

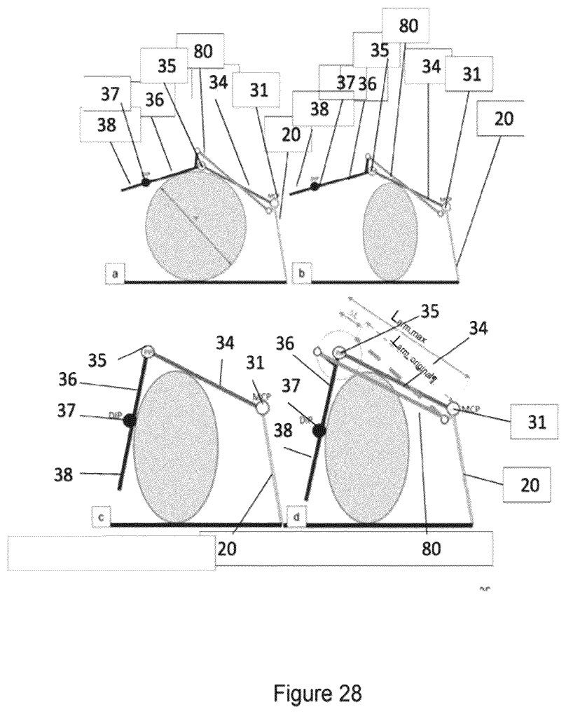

FIG. 28 shows a concept comparison of object adaptivity;

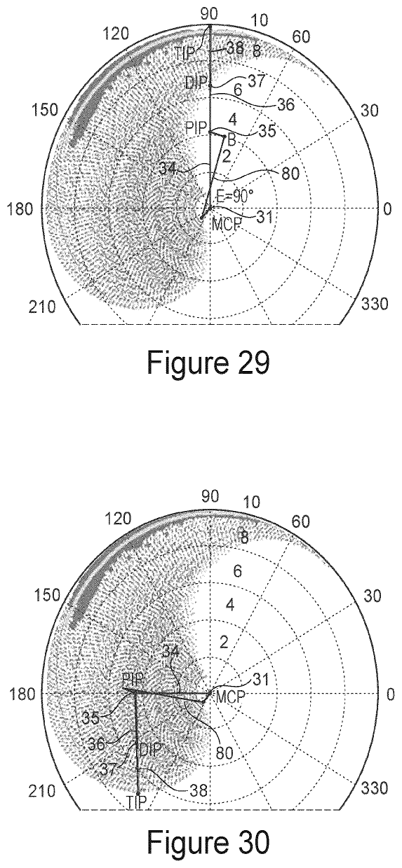

FIGS. 29 and 30 show trajectories of the fingertip;

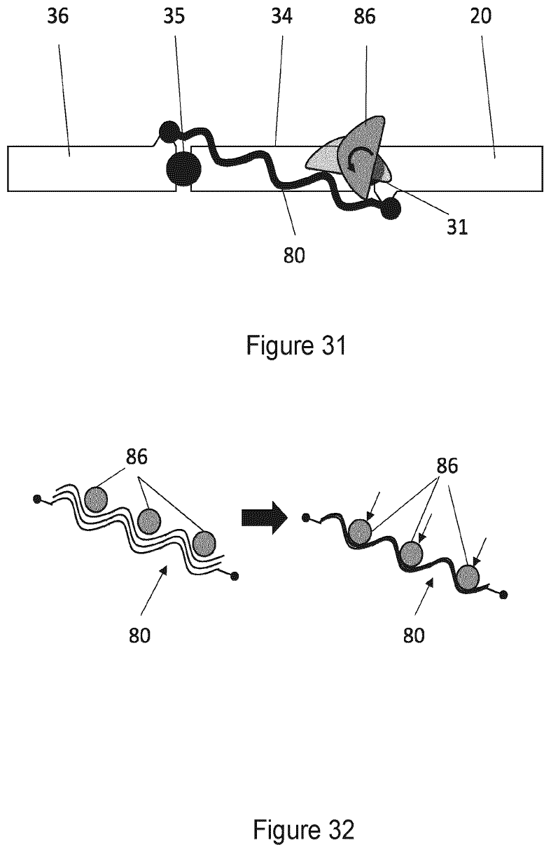

FIG. 31 shows a representation of a deformation element on a coupling rod; and

FIG. 32 shows different settings of the deformation element.

FIG. 1 shows a hand orthosis 10 in the fitted state with a support part 20, which is arranged above the back of the hand. Extending from the support part 20 in the proximal direction along the forearm, there is a forearm receiver 60 which extends spirally from the back of the hand onto the inner side of the forearm. The forearm receiver 60 is secured to the forearm via fastening means, for example via cuffs, straps or the like, which are fastened for example via a hook-and-loop fastener, a clasp or a buckle to the forearm. The support part 20 and the forearm receiver 60 in the exemplary embodiment represented are formed integrally and in particular made from a plastic, a fiber-reinforced composite material and optionally also from a light metal. There is also the possibility that the support part 20 with the forearm receiver 60 is manufactured separately and connected to one another and secured. Formed distally on the support part 20, there is a finger portion 30 which extends from the end of the back of the hand over the metacarpophalangeal joint as far as the fingertip region. The finger portion 30 in the exemplary embodiment represented is formed in one piece and formed integrally with the support part 20. The finger portion 30 does not extend over the entire width of the fingers but tapers obliquely in the distal direction, which may be seen in the central representation of FIG. 1. A finger receiver 32, via which the fingers are coupled to the finger portion 30, is formed at the distal end region of the finger portion 30. The finger receiver 32 makes it possible that the fingers can follow a deformation movement in the extension direction. Since the finger portion 30 is arranged dorsally, or on the upper side of the fingers, a flexion movement of the finger portion 30 also leads to a flexion of the fingers by the transmission of compressive forces. The finger receiver 32 may for example be formed as a pocket or a plurality of pockets, as a loop or loops, buckles, straps or other fastening elements that make it possible to secure the finger or the fingers to the finger portion 30. A thumb receiver 33, which is formed on a thumb portion, is likewise arranged on the support part 20. The thumb portion may also be formed separately and fastened to the support part 30. The thumb receiver 33 also secures the thumb to the thumb portion, which may for example be formed integrally with the support part 20, in a similar way to the finger portion 30.

A leaf spring 160 is mounted displaceably on the upper side of the support part 20. The leaf spring 160 is mounted longitudinally displaceably on the upper side of the support part 20 on a guide element, which at the same time constitutes a fixing device 50. The guide element and the fixing device 50 are arranged in an elongate hole or in a slot of the leaf spring 160. The leaf spring 160 is a bent spring element and is displaced in the proximal direction or distal direction and secured via the fixing device 50 in the desired setting. If the leaf spring 160 is in the position displaced forward most in the distal direction, the distal regions of the finger portion 30 are subjected least to a spring force. The finger portion 30 has a substantially flexed setting in the normal setting, without force action by an actuator 40.

The left and right representations of FIG. 1 represent a variant in which the actuator 40 is arranged at the proximal end of the forearm receiver 60. The actuator 40 is connected to the finger portion 30 or to the leaf spring 160 via a force transmission device in the form of a force transmission element 110, which in the exemplary embodiment represented is formed as a control cable or a strap. If the actuator 40, which is formed as a lever, is for example displaced backward, the force transmission device or the force transmission element 110 is tensioned. Depending on where the force transmission device or the force transmission element 110 is fastened, either the distal region of the finger portion 30 is pulled upward and extended or the leaf spring 160 is displaced in the proximal direction. If the force transmission device or the force transmission element 110 is fastened to the finger portion 30, the desired setting is secured via the fixing device 50. Because of the elastic configuration of the finger portion 30 or of the leaf spring 160, in the case of a released fixing device 50 a flexion movement then automatically takes place. The actuator 40 may also be displaced between two dead-point positions, so that a fixing device 50 may be omitted when only two positions of the finger portion 30 are intended to be occupied.

If the force transmission device or the force transmission element is mounted on the displaceably mounted leaf spring 160, the latter slides along the curvature when the actuator 40 is released into a position that is displaced forward in the distal direction. A force is in this case exerted on the distal region of the finger portion 30, so that a flexion takes place. If the leaf spring 160 is retracted, i.e. moved in the direction of the forearm receiver 60, the leaf spring 160 slides back on the upper side of the support part 20 and is stretched because the leaf spring 160 is limited upward by the guide element and the fixing device 50 in the case of a backward displacement movement on the guide element and the force action point of the leaf spring 160 is shifted proximally. In the desired setting, the actuator 40 and therefore also the deformed leaf spring 160 are retained via the fixing device 50. The fixing device 50 can bring about locking in discrete steps, for example by catch elements with form-fit locking, when the fixing device 50 is displaced for example downward in the direction of the support part 20. A rotation of the fixing device 50 for the form-fit locking or clamp locking is also produced in some embodiments. The actuation of the actuator 40 takes place manually, and the movement of the leaf spring 160 may also take place directly without a lever as the actuator, in which case the leaf spring 160 is then at the same time the actuator. In order to facilitate the activation, in one embodiment grip elements, form-fit elements or the like (not represented) are arranged on the leaf spring 160 on the rear side in order to facilitate the displacement and movement of the leaf spring 160 in the distal direction or proximal direction. If the leaf spring 160 is moved backward, an extension of the finger portion 30 and therefore also of the fingers in the finger receiver 32 takes place; conversely, a flexion is brought about by a displacement movement in the distal direction.

The right representation of FIG. 1 represents a variant in which the actuator 40 has an actuation element in the form of a lever, which is arranged at the proximal end of the forearm receiver 60. The actuation element is coupled via a force transmission device or a force transmission element 110, which in the exemplary embodiment represented is formed as a control cable or a strap, to the leaf spring 160 or to the finger portion 30, and in particular to the finger receiver 32, and extends the finger portion 30 when the force transmission device 110 is tensioned. In the central representation, neither an actuation element nor a force transmission device is fastened to the forearm receiver 60. The actuator is then, for example, the leaf spring element 160 on the support part 20.

FIG. 2 represents a variant of FIG. 1, in which a damper 90 is arranged on the force transmission element 110 and the fixing device 50 underneath on the actuator 40 performs retention of the actuator 40 in the desired position. The finger receiver 32 is formed in the embodiment according to FIG. 2 as a pocket or cap, in which all fingers except the thumb can be received. The force transmission element 110 runs from the back of the hand in the support part 20 in a channel through the damper 90 to the actuation element of the actuator 40 at the proximal end region of the forearm receiver 60. The damper 90 prevents a spontaneous displacement of the actuator 40 in the case of relaxation of the force transmission element 110, or snapping back of the extended finger portion 30 in the case of relaxation of the leaf spring 160 with a loosened fixing device. It may be seen in the right representation of FIG. 2 that the force transmission element 110 is fastened to the actuation element of the actuator 40. In the position represented, the actuator 40 is in an almost maximum distal setting with a relaxed force transmission element 110 and holds the hand orthosis 10 in the desired basic positions. So that an extended setting can be occupied, the actuation element is displaced, and in the exemplary embodiment represented the lever is turned backward in the proximal direction. The force transmission element 110 fastened to the distal end of the finger portion 30 is thereby tensioned and the finger portion 30 is extended. The fingers extend and can be retained via the fixing device 50 in the desired position. Since the force transmission element 110 is fastened to the displaceably mounted leaf spring 160, the leaf spring 160 is displaced backward in the case of an actuation of the actuator 40, and either the leaf spring 160 is stretched and brings about an extension or, in the case of a rigid leaf spring 160, the finger portion 30 is curved and carries out a flexion. There is likewise a thumb receiver 33 in the hand orthosis 10 according to FIG. 2, which is arranged mobile on the support part 20 and can be fixed in the respectively desired position. The thumb is then respectively in the adjusted setting of the thumb receiver 33 relative to the support part 20, so that the respectively desired type of grip can be produced in the case of a flexion movement or extension movement of the finger portion 30.

The finger portion 30 itself may be elastically formed in order to be able to resume the respectively adjusted initial position after the cessation of external forces, which for example are applied via the actuator 40. This leads to a spring action via the finger portion 30 for the springs. The spring action is adjustable; for example, a displacement of the leaf spring 160 may be brought about in the direction of the finger portion 30 or away from the finger portion 30, which may take place by a modifying mechanism. As soon as the fixing device 50 has released the pretensioned state, the orthosis returns into the initial state. The damper 90 prevents an excessively rapid flexion movement or, in the converse case, an excessively rapid extension movement, wherein the damping rate may be adjustable. The damper 90 may be configured as a brake or another resistance device, in order to influence the restoring movement.

FIG. 3 represents a further variant of the orthosis 10 in a side view. Here as well, the actuation takes place via the actuator 40 in the form of a lever, which is arranged on the lower side of the forearm receiver 60 and is coupled via the force transmission element 110 to the finger portion 30. The leaf spring 160 at the side of the finger portion 30 is represented as being loosened from the finger portion 30. The curvature that a flexion movement brings about can be seen. The finger receiver 32 secures the fingers to the finger portion 30, the finger receiver 32 being configured so that the leaf spring 160 has no negative influence on the haptics or the grip. Via the damper 90 (not represented), the restoring movement is damped in the case of a release of the fixing device 50, for example when the actuator 40 is displaced from the tensioned, extended setting, in which it is in a dead position, into the forward position depicted in order to occupy the basic setting. The actuation of the actuator 40 takes place by the contralateral hand, so that a purely manually operable hand orthosis 10 can be provided. The finger portion 30 is formed elastically together with the support part 20, and the thumb receiver 33 is fastened to the support part 20, modifiably with respect thereto.

FIG. 4 represents a variant of the hand orthosis without a thumb portion 33; the fixing to the forearm takes place via spiral guiding of the forearm receiver 60 around the forearm and securing via a strap. A guide for the force transmission element 110 is incorporated inside the forearm receiver 60, or fastened thereto.

FIG. 5 represents a variant of the orthosis 10 that has a thumb receiver 33, which is passively positionable in respect of its setting. In contrast to the form of configuration according to FIG. 4, the forearm receiver 60 is guided from the support part 20 laterally around the forearm in the direction of the proximal direction; in FIG. 4 the forearm receiver 60 is guided medially at the side of the thumb onto the lower side of the forearm. Formed inside the forearm receiver 60, there may be a pliable or flexible, optionally elastic region that enables bending of the forearm receiver 60 in the region of the transition from the back of the hand to the lower side of the forearm, so that adaptation to different diameters is possible as shown in the upper right representation of FIG. 5. In this way, the radii of curvature of the forearm receiver 60 may be adapted.

A hand orthosis as represented in FIGS. 1 to 5 does not require any electronic control or an energy accumulator. Making it from plastic and/or a fiber-reinforced composite material provides a high load-bearing capacity with a low weight. The hand orthosis 10 is readily adaptable to different patients and enables positioning of the hand in the respectively desired position. By the adjustment of the elastic restoring force, it is possible to carry out a certain preadjustment of a closing force when the basic setting is intended for example to be the closed setting. The damper 90 or the damper system regulates the speed of the restoring movement and prevents the hand orthosis from spontaneously opening or snapping shut. The initial setting of the fingers relative to the back of the hand, or of the finger portion 30 with respect to the support part 20, may be either open or closed, wherein the elastic configuration takes place either by manufacturing the support part 20 together with the finger portion 30 and/or by corresponding mounting and fitting of a separate spring mechanism or a spring. After the finger portion 30 has been brought out of the initial setting, the use setting is held by the fixing device 50. Only when the fixing device 50 is deactivated, for example by a mechanical trigger which is fitted at an ergonomically expedient position, does the finger portion 30 and therefore also the hand close or open again. The respective use setting may be freely selected and may lie either between two discrete states, which define an open or closed setting, or in discrete or continuous steps between them. The fixing takes place for example by a ratchet mechanism or by a clamp mechanism. If a thumb receiver is provided, it may be secured to the support part 20 in a defined setting with respect to the other fingers or the hand; alternatively, the thumb receiver is passively modifiable and can be fixed in different adjustable settings.

A variant is represented in FIG. 6, in which various possible uses of the hand orthosis are shown. In the left representation of FIG. 6, the hand orthosis 10 is in the open setting, for example in the initial setting, and may be used to grip objects without closing the hand. The right representation of FIG. 6 shows a state in which the hand orthosis 10 is in a closed use setting. In order to reach this use setting, a form-fit element 130 is provided as a part of the actuator 40 and is formed or arranged in the form of a loop at the proximal end of a comparatively stiff proximal actuator portion 150. The form-fit element 130 or the loop engages with a counter element 230, which is secured to the torso of the user. In the exemplary embodiment represented, the counter element 230 is formed as a hook or as a projection which is fastened to the hip, for example to a belt. In order to actuate the hand orthosis 10, the form-fit element 130 is brought up to the counter element 230 and hooked into it, and the hand with the hand orthosis 10 is moved forward. In this way, a tensile force is exerted via the actuator 40 and the hand orthosis is moved from the opened initial setting into the closed use setting, so that an object can be held in the closed hand. By activating a fixing device, the closed hand posture is then maintained. If no fixing device is activated, the hand opens when the hand with the hand orthosis 10 moves in the direction of the counter element 230, since the tensile force of the actuator 40 is absent and the initial setting is occupied again because of the elastic mounting or the elastic configuration of the finger portion 30. Here as well, a damping device is advantageously arranged in the hand orthosis 10 or in the forearm receiver 60 in order to slow or adjust the restoring movement.

FIG. 7 represents the process of hooking in the form-fit element 130. A loop that is held open is introduced into the counter element 230, which is formed as a hook, and the hand is moved forward. This is indicated by the arrow in the right representation of FIG. 7. By the application of a tensile force, the hand orthosis 10 is closed, which is indicated by the curved arrow at the hand orthosis 10. A correspondingly reversed movement takes place when unlocking the form-fit element 130. Such an actuation device or such an actuating mechanism enables the gripping and fixing of objects in a hand provided with the hand orthosis 10, particularly in the case of standing activities when persons with hand paralysis but in other regards good mobility in the arm or shoulder as well as in the torso wish to carry out activities. Other devices for producing an opposing bearing for the actuator via the form-fit element on the torso are possible, for example by a hook on the form-fit element 130, by a ball which is arranged at the end of a cord or of a rod and can be introduced into a corresponding cup-shaped ball socket, or by other form-fit devices.

A further configuration of the hand orthosis is represented in FIGS. 8 and 9. FIG. 8 shows a detail view of a hand orthosis 10 with the support part 20 and finger parts 34, 36, which are arranged distally thereon and are mounted on one another pivotably about pivot axes 31, 35. The support part 20 is for example secured to the upper side of the back of the hand and is fixed thereto via a strap, which reaches around the wrist or around the forearm. A first disk-shaped receiver as a first finger part 34 is pivotably mounted distally with respect to the support part 20. The pivoting takes place about a pivot axis 31 which essentially corresponds, or is intended to correspond, to a compromise axis of rotation of the metacarpophalangeal joints. The proximal finger part 34 extends from the metacarpal bone in the distal direction over the first phalanx, or phalanx proximalis, of a natural hand. The second, distal finger part 36 extends over the other two phalanges, i.e. the phalanx medialis and the phalanx distalis.

Instead of an integral configuration of the finger portion 30 as described above, there is a segmental configuration of the finger part 30, in the exemplary embodiment represented a bipartite configuration. The attachment of the actuator, or of the force transmission device, takes place dorsally. For example, a control cable which is guided via the proximal finger part 34 and via the support part 20 to the actuator, and which extends the finger part 30 in the case of actuation of the actuator, is fastened to the distal finger part 36. The basic setting is adjusted and maintained by spring elements that are active about the pivot axes 31, 35, for example in the form of torsion springs or coil springs, although a leaf spring may alternatively be arranged on the back of the hand in a similar way as in FIGS. 1-5. FIG. 9 shows the hand orthosis in a fitted state, the support part 20 having a forearm receiver 60 which is fixed to the lower side of the wrist via a strap or hook-and-loop fastener. The finger portion 30 has two finger parts 34, 36, which are connected articulated to one another via the corresponding axes 31, 35. The control cable system via the actuator may be provided with a spring pretension and optionally with a damper. FIG. 9 indicates the torsion springs 163 that act about the joint axes 31, 35, although the torsion springs 163 may also be configured as elastomer elements. As an alternative to the configuration with two pivot axes 31, 35, the finger portion 30 may also be formed with three finger parts which can be displaced about three pivot axes relative to the support part 20.

Such a configuration is represented in FIG. 10, in which the individual finger parts 34, 36, 38 are connected to one another via film hinges or leaf springs while forming joint axes 31, 35, 37. The force transmission element 110 in the form of a strap is guided along the back of the hand, as may be seen in the right representation of FIG. 10. The individual finger parts 34, 36, 38 correspond to the phalanges; the film hinges may be elastically pretensioned or formed as leaf spring elements and, besides providing the pivot axes 31, 35, 37, may also at the same time bring about the elastic mounting of the finger elements 34, 36, 38 on one another. Depending on the configuration of the connecting elements between the finger parts 34, 36, 38, there is an extended or opened hand posture as the basic setting or a flexed or closed hand posture. The right representation of FIG. 10 shows the thumb receiver 33, to which the thumb can be fastened. The thumb receiver 33 is passively adjustable, so that different types of grip can be implemented. If a tensile force is applied by the actuator from the initial setting according to FIG. 10 via the force transmission element 110, the spring elements between the finger parts 34, 36, 38 are tensioned and force is stored therein. In order to prevent an unintended closure movement, the force transmission element 110 is fixed in the respectively desired position. In order to bring about a restoring movement into the initial setting, the fixing device is deactivated so that the hand orthosis closes again, with a damper slowing the closure movement in a desired manner. The damper advantageously acts on the force transmission element 110.

FIG. 11 shows a configuration with a damper 90 in a perspective representation. The actuator 40, for example as a pusher with a grip element, is arranged on the forearm receiver 60. Projections or form-fit elements, in which a fixing device 50 can engage with a form fit, may be arranged on the pusher. For example, a tensile movement may be always possible, which may easily be produced mechanically by a corresponding orientation of sawtooth elements. The fixing device 50 is pretensioned in the direction of a closure movement, so that the respectively proximal setting of the actuator 40 is fixed in the case of retraction of the actuator 40 and tensioning of the spring elements between the finger parts. If the actuator 40 is not moved further in the proximal direction, the tensile stress inside the force transmission element because of the restoring action of the spring elements remains. If the fixing device 50 is deactivated, for example by the elastically mounted fixing element 50 being unlocked, release takes place so that the actuator 40 can be pulled back in the distal direction. This restoring movement is influenced and slowed by the damper 90, which is represented schematically, in order to avoid rapid snapping back during the closure movement. The closure movement due to the release of the fixing device 50 is represented by the arrow movement, so that the thumb receiver 33 and finger receivers 32 move toward one another. The setting of the thumb receiver 33 is in this case freely selectable.

FIG. 12 represents different settings of the hand orthosis. The respectively upper left representation represents the initial setting with a closed hand and the corresponding hand orthosis. If the actuator is actuated and the force transmission device or the force transmission element 110 moves backward, the finger portion 30 is extended and the hand is opened. In the lower left representation, the hand is in the extended setting that is provided with the spring pretension. The lower right representation shows that a slow, damped restoring movement via the damper 90 in the case of a release of the fixing device and a restoring movement are carried out because of the relaxation of the springs. The loading of the springs takes place in the left representation by a linear movement of the actuator, and in the right representation by a tilting movement of a lever.

FIG. 13 represents the different positions of the thumb receiver 33 relative to the finger part, or the finger receiver 32. In the left representation, the thumb is in a splayed setting so that a pinch grip takes place in the closed setting. The modification takes place passively via a thumb portion joint 330. In the right representation, the thumb is parallel to the proximal phalanx of the index finger in order to enable a so-called power grip.

FIG. 14 shows a variant of FIG. 9, in which two finger parts 34, 36 are likewise formed as a finger portion 30 with finger receivers 32. Furthermore, the thumb receiver 33 is mounted articulated and pivotably on the support part 20. The elastic mounting takes place via a spiral spring 162. The elastic mounting of the finger parts 34, 36, or of the finger portion 30, takes place via a spiral spring 162 as a tension spring element, which is arranged on the distal end of the distal finger part 36 and is secured via a tension element to the support part 20. Bending of the finger portion 30, or of the finger parts 34, 36, is carried out via the actuator 40 which is secured to the support part 20, via the force transmission device or the force transmission element 110, the structure and mode of operation of which are explained in more detail. The actuator 40 may be secured replaceably and freely configurably to the support part 20 or to the forearm receiver 60, which is not represented. Owing to the modular configuration of the actuator 40 and the replaceability, it is possible to adapt the adaptation of the orthosis to the respective user and the respective situations of use. Furthermore, it is possible to define different initial settings, for example the closed setting or the open setting, as an initial setting. Fixing devices and release devices may also be integrated in the actuator 40, as well as a damper in order to influence the movement in the case of relaxation of the pretensioned springs. The actuator itself, for example when it is configured as an electric motor, may constitute a damper in generator operation.

The actuator 40 may be configured to be driven both by a motor and purely manually. If the actuator 40 is activated, the control cable or the force transmission element 110 is pulled in the proximal direction and therefore brings about bending of the finger parts 34, 36 about the respective pivot axis and therefore flexion and closure of the hand. The thumb with the thumb receiver 33 either may be held fixed in the respectively adjusted setting or is actuated via other actuation devices or the actuator 40. The control cable or the force transmission element 110 is tensioned against the elastic restoring force of the spiral spring 162. If the fixing device is deactivated, the tensioned spring 162 relaxes and the finger portion 30 returns into the initial setting.

FIG. 15 schematically represents the actuator 40. The actuator 40 has two clutches 100, which are coupled to a force accumulator 105 and via force transmission elements 110 to the distal finger part 36, or the thumb receiver 33. The clutches 100 are therefore part of the fixing device 50 and cause the finger parts 34, 36, or the thumb receiver 33, to be held in the desired setting after an actuation of the actuator 40 and the displacement from the initial setting into the use setting. Arranged on the fixing device 50, there is a release element 501 that can deactivate the fixing device 50. The detailed structure will be explained with the aid of FIG. 18. By the actuators 40, the respective control cables or force transmission elements 110 are rolled up; a coil spring 161 as a force accumulator 105 is assigned to the respective rolls and is tensioned in the case of rolling up and therefore tensioning of the springs 161, 162.

In the desired position, the actuator is blocked by the fixing device 50 and secured against rotating back. By actuation of the release element 101, a freewheel is unlocked so that both the spiral spring 161 and the coil springs 162 relax.

In FIG. 16, the drive 40 is configured as an electric motor. In the case of actuation of the actuator 40, the hand orthosis 10 is closed against the elastic restoring force of the springs 162. The electric motor is arranged as a drive or actuator 40 directly on the fixing device 50 with the clutches.

In FIG. 17, an actuator arranged at a distance from the fingers is provided, and is not arranged directly on the support part 20 but, for example, is positioned as a motor on the forearm receiver. The actuator may also be configured as a lever, pusher, sliding element, rotating element or the like and may be coupled via a force transmission device or a force transmission element 110 to the fixing device 50.

The fixing device 50 is represented in a more detailed representation in FIG. 18. The winding spring 161, two winding disks 502 and a freewheel 504 are mounted on a common axis. A blocking ring 503, which is rigidly coupled to the outer ring of the freewheel 504, is arranged on the outer circumference of the freewheel 504. The winding spring 161 is secured both to the housing and to the axis. The winding disks 502 receive the force transmission element 110, which is formed in particular as a cable or Bowden cable. The fixing device 50 makes it possible that the finger parts 34, 36 and the thumb receiver 33 are movable in a movement direction, i.e. flexion or extension. The force transmission elements 110 are in this case rolled up by the winding spring 161. By the release element 501, which can be introduced into recesses in the blocking ring 503, the winding disks 502 remain in the adjusted position. Only when the blocking ring 503 is released by the actuation of the release element 501 does the spring 161 relax together with the springs 162 and the thumb receiver 33 as well as the finger parts 34, 36 are moved back into the initial setting.

A further configuration of the hand orthosis with a support part 20 and with a multisegmented finger portion 30 is shown in FIGS. 19 to 24. FIG. 19 shows a skin-colored support part 20 with two finger parts 34, 36 arranged articulated thereon, which are configured in a similar way to the embodiment according to FIGS. 8 and 9. In addition or as a supplement to the torsion springs that supply the finger parts 34, 36 with a spring pretension relative to one another and relative to the support part 20, a coupling rod 80 is arranged between the support part 20 and the distal finger part 36. In the exemplary embodiment represented, the distal mounting point 83 of the coupling rod 80 is arranged dorsally, i.e. on the upper side and therefore above the pivot axis 35, on the distal finger element 34. The proximal mounting point 81 of the coupling rod 80 on the support part 20 lies below the pivot axis 31, i.e. closer than the pivot axis 31 to the hand. The coupling rod 80 therefore overtensions the proximal finger part 34 and two joint axes 31, 35. In the exemplary embodiment represented in FIG. 19, a coupling rod 80 is represented only on the medial side, although corresponding receivers in the form of bolts or projections may also be arranged or formed on the lateral side on the support part 20 and on the distal finger part 36 in order to fit a second coupling rod 80 there.

FIG. 20 represents the individual components schematically in a side view. The force transmission element 110 is likewise represented, which is guided through the proximal pivot axis 31 and is secured below the distal pivot axis 35 to the distal finger part 36. By the connection of the finger parts 34, 36 via the coupling rod 80, it is possible to ensure a physiological movement of the fingers. In particular, a physiological finger flexion as well as a finger extension may be produced with only one force action. The coupling rod 80 is elastically configured so that, in addition to its function as force transmission between the support part 20 and the distal finger part 36, the coupling rod 80 has the possibility that it can be elongated and contracted. This provides an enhanced functionality of the hand orthosis 10. By the possibility of the coupling rod 80 assuming different lengths, the individual finger parts 34, 36 can be moved independently of one another. The distal finger parts can also yield separately under distal loads, which protects the components. In this way, a natural movement of the fingers coupled to the finger parts 34, 36 is possible, which avoids damage to the joints, muscles and tendons due to unsuitable guiding. In particular, adaptive gripping is possible with such a configuration.

FIG. 21 shows a setting of the individual components with respect to one another, a deformation element 310 in the form of a cam disk, which acts on the force transmission element 110, being arranged on the proximal finger part 34. By the deformation element 310 in the form of the cam disk, the course of the force transmission element 110 can be manipulated. In the representation according to FIG. 21, the force transmission element 110 or the control wire runs through the proximal joint axis 31 and generates only a moment about the distal pivot axis 35, so that only the distal finger part 36 flexes. In FIG. 22, the deformation element 310 is rotated so that it acts on the force transmission element 110. It therefore runs at a relatively large distance below the pivot axis 31 or the point of rotation about the pivot axis 31, and therefore generates a moment not only about the distal pivot axis 35 but also about the proximal pivot axis 31.

FIG. 23 represents an individual representation of the coupling rod 80 which was used in the embodiments of FIGS. 21 and 22. The coupling rod 80 has an S-shaped, sinusoidal central region that is arranged between the proximal and distal mounting points 81, 83, which are configured as axis receivers. The coupling rod 80 may be formed from a plastic or another elastic material, and allows both transmission of tensile forces and compressive forces and elongation or contraction with simultaneous force transmission.

The coupling rod 80 mounted on the support part 20 as well as the distal finger part 36 with the finger receiver 32 are represented in FIG. 24.

FIG. 25 represents two coupling rods 80, which are configured in a similar way to that of FIG. 23. The coupling rods 80 likewise have two mounting points in order to be able to fasten the coupling rod 80 to one of the components of the hand orthosis or hand prosthesis, for example distally with respect to the PIP and proximally with respect to the MCP. The coupling rod 80 of FIG. 25 is formed segmentally, the resilient or elastic central region being formed as a separate component which is replaceably fastened at the rectilinearly configured end regions to the bearing points. The right coupling rod 80 has a strengthened central region, so that a greater spring resistance is provided against deformation. The elastic region is configured, in the exemplary embodiments represented, to be undulating, particularly in the form of a sinusoidal wave.

FIG. 26 represents two states of a coupling rod 80 with a linear actuator 82. In the left representation of FIG. 26, the linear actuators are in a central setting that enables displacement of the two mounting points both away from one another and toward one another. The linear actuator 82 is configured in a similar way to a hydraulic damper or a pneumatic spring, and has a housing as a receiver for a piston-like end piece. A linear movement of the piston-like end piece inside the housing is possible, so that an effective shortening or lengthening of the piston rod 80 can be adjusted. When the linear actuator is configured as a pneumatic spring, the end piece subdivides the cylindrical housing into two chambers so that a compression in a chamber takes place in the case of both a contraction and an elongation of the coupling rod 80. Alternatively or in addition, at least one spring is arranged in or on the linear actuator in order, after a displacement of the two components of the coupling rod 80 with respect to one another, to move them back into an initial setting after an external load ceases. In the right representation, the linear actuator 82 is in the maximally deployed position from which a restoring then takes place when the external force ceases.

FIG. 27 shows an embodiment of the coupling rod 80 with a length limitation 84. In the left representation of FIG. 27, the coupling rod 80 is in a relaxed state or in a contracted state in which the length limitation 84, which is formed for example as a wire that spans the elastically deformable region of the coupling rod 80, is relaxed. The wire or the length limitation 84 is in particular rigid in tension and at least flexible, so that after an elongation of the coupling rod because of a tensile force the length limitation is tensioned, which is shown in FIG. 27 in the right representation. If the coupling rod 80 is in the state of the right representation of FIG. 27, it acts as a rigid coupling rod. After a tensile force between the mounting points ceases, the coupling rod returns because of the resiliency of the elastic region back into the initial state, for example into the state of the left representation of FIG. 27.

FIG. 28 represents four schematic representations of the object adaptivity of the individual prosthetic fingers or orthotic fingers. The upper left representation of FIG. 28 represents a support part 20 with a proximal phalanx 34, a medial phalanx 36 and a distal phalanx 38. A rigid coupling rod 80 is arranged between the support part 20 and the medial phalanx 36. In the case of bending of the proximal finger part 34 about the pivot axis 31, forced bending about the pivot axis 35 of the PIP takes place because of the coupling rod 80. With this configuration, it is readily straightforward to grasp a cylindrical object. In the upper right representation of FIG. 28, the object to be gripped is not cylindrical but has an oval cross section. The entire joint system is blocked since direct contact of the object takes place with the proximal finger part 34, so that further bending and flexion about the pivot axis 35 cannot take place despite the coupling rod 80.

The lower left representation of FIG. 28 represents the object adaptivity without a coupling drive via a coupling rod; in contrast thereto, the lower right representation shows an orthotic finger or prosthetic finger having a coupling drive with a coupling rod 80. The coupling rod 80 is variable in length, specifically by the length AL, which leads to automatic and improved contact of the medial phalanx 36 and improved grasping by the distal finger part 38.

FIGS. 29 and 30 show physiological trajectories of the fingertips: in FIG. 29 the finger is in the extended setting, and in FIG. 30 it is in the 90° curved setting, in which the proximal finger part 34 is flexed about the pivot axis 31 by 90°. Via the coupling rod 80, pivoting of the medial finger part 34 takes place about the pivot axis 35; the distal finger part 38 remains extended and does not pivot about the pivot axis 37. The trajectory of the fingertip of the orthosis or prosthesis is in this case assimilated to the natural trajectory of a fingertip.

FIG. 31 represents a schematic representation of a prosthetic finger or of an orthotic element with a support part 20, a proximal finger part 34 and a distal finger part 36. The individual finger parts 34, 36 are mounted articulated about pivot axes 31, 35 on the component that is respectively proximal thereto. In the exemplary embodiment represented, a deformation element 86 is arranged on the proximal finger part 34, and can be pivoted together with the proximal finger part 34 about the pivot axis 31 in the case of a displacement. The deformation element 86 is arranged fixed in rotation on the proximal finger part 34. The coupling rod 80 is arranged, as shown in FIGS. 22 and 24, crossing the joint on the support part 20 and the distal finger part 36. The mounting points of the coupling rod 80 lie on different sides of a connecting plane between the pivot axes 31, 35. The coupling rod 80 or the coupling element is formed resiliently and has an undulating shape. In the case of pivoting of the proximal finger part 34, the deformation element 86 acts on the coupling rod 80 and brings about or reinforces a deformation of the coupling rod 80, so that a variation of the spring stiffness or a variation of the effective length, i.e. shortening or lengthening of the distance between the mounting points, can take place. In the exemplary embodiment of FIG. 31, the coupling rod 80 is formed solidly and the deformation element 86 is formed as a cam, which slides along the surface of the coupling rod 80.

FIG. 32 represents a variant of the deformable coupling rod 80 coupled to a deformation element 86. The coupling rod 80 likewise has an undulating shape and is formed in multiple layers. In the left representation of FIG. 32, the deformation element 86, which may have a plurality of action regions, is positioned out of engagement with two spring layers of the coupling rod, so that only one spring or one spring layer of the coupling rod 80 is active. The spring is correspondingly soft, or the coupling rod 80 is correspondingly pliant. In the right representation of FIG. 32, the coupling element 86 is engaged with the individual spring layers and presses them onto one another, so that three springs act in total since a compression or tensile movement that is applied to the bearing points of the coupling rod 80 is transmitted by the undulating shape. The action regions of the deformation element 86 bring the three spring layers in the coupling rod 80 into contact with one another, as a result of which the spring stiffness is increased overall, so that the spring stiffness is influenced by the deformation element.

The configuration of the force transmission with the coupling rod, as is represented and described in the figures, is not restricted to the application of hand orthoses. For instance, such a configuration may also be used for prosthetic applications or alternatively in robotics. Instead of the support part 20, a base body is provided in the case of a prosthetic hand, which corresponds to the metacarpus and in which drive devices are generally arranged. Instead of the finger parts 34, 36, prosthetic fingers with corresponding joints, components and prosthetic finger parts are arranged pivotably on this base body. Likewise, in one embodiment, three finger parts are arranged articulated to one another, corresponding to FIG. 10. The finger parts are then respectively coupled via the coupling rod 80, which are arranged opposite one another across on two joints with different positioning of the respective fastening points on different sides of the joint axes. The coupling rod 80 spans the phalanx located between the joint axes, for example the proximal phalanx between the support part and a distal phalanx, and therefore provides a direct, integral connection between the support part and the distal phalanx. The coupling rod 80 in the embodiments represented is not driven, although in principle the coupling rod 80 may also be coupled to a drive.

### LIST OF REFERENCE SIGNS

10—hand orthosis
20—support part
30—finger portion
31—pivot axis
32—finger receiver
33—thumb part
34—finger part
35—pivot axis
36—finger part
37—pivot axis
38—finger part
40—actuator
44—actuation element
50—fixing device
60—forearm receiver
70—adjustment device
80—coupling rod
81—proximal mounting point
82—linear actuator
83—distal mounting point
84—length limitation
86—deformation element
90—damper
100—clutch
105—force accumulator
110—force transmission element
120—motor
130—form-fit element
140—grip element
150—actuator portion
160—leaf spring
161—spiral spring
162—coil spring
163—torsion spring
230—counter element
235—fastening device
310—deformation element
501—release element
502—winding disk
503—blocking ring
504—freewheel

1. A hand orthosis or hand prosthesis, comprising:
a support part;
at least one finger portion arranged distally on the support part, wherein the at least one finger portion is mounted pivotably relative to the support part wherein the at least one finger portion is formed segmentally from a plurality of finger parts that are mounted articulated to one another;
a coupling rod coupled to a distal finger part of the plurality of finger parts and wherein the coupling rod is coupled to either a proximal finger part of the plurality of finger parts or to the support part, wherein the coupling rod crosses at least two joints between finger parts of said plurality of finger parts and is configured to transmit force, wherein the coupling rod is mounted in a first case on a palmar side and in a second case on a dorsal side the respect to a respective pivot axis of the at least two joints.

2. The hand orthosis or hand prosthesis as claimed in claim 1, further comprising an actuator coupled at a distal end of the actuator to the at least one finger portion and displaces the at least one finger portion from an initial setting into a use setting.

3. The hand orthosis or hand prosthesis as claimed in claim 1 further comprising a thumb part arranged on the support part.

4. The hand orthosis or hand prosthesis as claimed in claim 3, wherein the thumb part is arranged modifiably on the support part.

5. The hand orthosis or hand prosthesis as claimed in claim 3 wherein the thumb part is mounted fixably on the support part in a use setting.

6. The hand orthosis or hand prosthesis as claimed in claim 3 wherein the thumb part is elastically configured or elastically mounted on the support part.

7. The hand orthosis or hand prosthesis as claimed in claim 1 wherein the at least one finger portion is elastically configured or elastically mounted on the support part.

8. The hand orthosis or hand prosthesis as claimed in claim 7, further comprising an adjustment device for adjusting a spring force is assigned to the at least one finger portion.

9. The hand orthosis or hand prosthesis as claimed in claim 3 wherein the at least one finger portion and/or the thumb part is configured as a leaf spring and/or is elastically mounted on the support part via a leaf spring, torsion spring, spiral spring, coil spring or an elastomer element.

10. The hand orthosis as claimed in claim 2 wherein the actuator is coupled proximally with respect to the support part to a form-fit element which is reversibly moveable into engagement with a correspondingly configured counter element.

11. The hand orthosis or hand prosthesis as claimed in claim 10, wherein the form-fit element is configured as at least one of a ring, a loop, a hook-and-loop fastener part, and a hook.

12. The hand orthosis or hand prosthesis as claimed in claim 1 wherein the plurality of finger parts are mounted elastically in an initial setting.

13. The hand orthosis or hand prosthesis as claimed in claim 1 wherein the coupling rod is elastically mounted or configured.

14. The hand orthosis or hand prosthesis as claimed in claim 1 wherein the coupling rod has a length limitation or a linear actuator mechanism.

15. The hand orthosis or hand prosthesis as claimed in claim 1 wherein the coupling rod is configured to be at least locally curved or undulating.

16. The hand orthosis or hand prosthesis as claimed in claim 1 wherein the coupling rod is assigned at least one deformation element which is arranged on a finger part of the plurality of finger parts and/or on the support part.

17. The hand orthosis or hand prosthesis as claimed in claim 2 wherein the actuator is rotatably or displaceably arranged or mounted on the support part.

18. The hand orthosis or hand prosthesis as claimed in claim 2 wherein the actuator is coupled to a clutch and to a force accumulator.

19. The hand orthosis or hand prosthesis as claimed in claim 2 wherein the actuator is configured as a replaceable module.

20. The hand orthosis or hand prosthesis as claimed in claim 2 wherein the actuator has, or is configured as, a force transmission element that transmits tensile forces.

21. The hand orthosis or hand prosthesis as claimed in claim 20, wherein the force transmission element is assigned a deformation element wherein the deformation element is arranged on a finger part of the plurality of finger parts and/or on the support part.

22. The hand orthosis or hand prosthesis as claimed in claim 2 further comprising a fixing device by which the at least one finger portion is held in a use setting, wherein the fixing device is coupled to the actuator or to the at least one finger portion.

23. The hand orthosis or hand prosthesis as claimed in claim 22, wherein a form-fit element is arranged or configured proximally with respect to the support part on a stiff actuator portion.

24. The hand orthosis or hand prosthesis as claimed in claim 2 wherein the actuator is assigned a damper for damping a restoring movement from the use setting into the initial setting.

25. The hand orthosis or hand prosthesis as claimed in claim 24, wherein the damper has an adjustable damping rate.

26. The hand orthosis as claimed in claim 1 wherein the at least one finger portion has at least one finger receiver for receiving at least one finger.

27. The hand orthosis as claimed in claim 3 wherein the thumb part has a thumb receiver for receiving a thumb.

28. The hand orthosis as claimed in claim 1 wherein the support part is configured to run above a back of a user's hand in a the-fitted state.

29. The hand orthosis as claimed claim 1 the support part protrudes proximally beyond a user's wrist in a fitted state and has a forearm receiver.

30. A system consisting of the hand orthosis or hand prosthesis as claimed in claim 1 and a counter element having a fastening device for securing to a torso or to an extremity of a user.
